(19) United States
(12) Patent Application Publication
(10) Pub. No.: US 2005/0267221 A1
(43) Pub. Date: Dec. 1, 2005

(54) Use of curcumin and analogues thereof as inhibitors of ACC2

(75) Inventor: C. W. Wellen, Houston, TX (US)

Correspondence Address: FULBRIGHT & JAWORSKI, LLP, 1301 MCKINNEY, SUITE 5100, HOUSTON, TX, 77010-3095, US

(73) Assignee: Research Development Foundation, Houston, TX (US)

(21) Appl. No.: 11/128,888

(22) Filed: May 13, 2005

Related U.S. Application Data
(60) Provisional application No. 60/571,467, filed on May 14, 2004.

(51) Int. Cl. A61K 31/12

(52) U.S. Cl. 514/688

(57) ABSTRACT

The present invention relates to the use of curcumin or analogues thereof as modulators of mitochondrial fatty acid oxidation. More specifically, compositions of curcumin or analogues thereof can be used to inhibit acetyl-CoA carboxylase 2 (ACC2) thereby promoting fatty acid oxidation. Yet further, the invention relates to the use of curcumin and analogues thereof to increase mitochondrial fatty acid oxidation thereby promoting weight loss and/or reducing fat accumulation.

### CROSS REFERENCE TO RELATED APPLICATION

This application claims priority to U.S. Provisional Application No. 60/571,467 filed May 14, 2004, which is incorporated herein by reference in its entirety.

### TECHNICAL FIELD

This invention relates in general to the field of metabolism and weight loss and/or weight management. More specifically, the invention relates to the use of curcumin or analogues thereof as inhibitors of acetyl-CoA carboxylase 2 (ACC2). Yet further, the invention relates to the use of curcumin and analogues thereof to increase mitochondrial fatty acid oxidation thereby promoting weight loss and/or reducing fat accumulation.

### BACKGROUND OF THE INVENTION

Acetyl-CoA carboxylase (ACC), a biotin-containing enzyme, catalyzes the carboxylation of acetyl-CoA to form malonyl-CoA, an intermediate metabolite that plays a pivotal role in the regulation of fatty acid metabolism (Wakil et al., 1958; Wakil et al., 1983; and Thampy, 1989). It has been found that malonyl-CoA is a negative regulator of camitine palmitoyltransferase I (CPTI, a component of the fatty-acid shuttle system) (McGarry et al., 1977; McGarry et al., 1997) that is involved in the mitochondrial oxidation of long-chain fatty acids. This finding provides an important link between two opposed pathways—fatty-acid synthesis and fatty-acid oxidation.

In prokaryotes, acetyl-CoA carboxylase is composed of three distinct proteins—the biotin carboxyl carrier protein, the biotin carboxylase, and the transcarboxylase (Moss et al., 1971). In eukaryotes, however, these activities are contained within a single multifunctional protein that is encoded by a single gene. In animals, including humans, there are two isoforms of acetyl-CoA carboxylase expressed in most cells, ACC1 (Mᵣ˜265,000) and ACC2 (Mᵣ˜280,000). ACC1 and ACC2 are encoded by two separate genes and display distinct tissue distribution (Wakil et al., 1983; Thampy et al., 1989; McGary et al., 1977; McGarry et al., 1997; Abu-Elheiga et al., 2000; Abu-Elheiga et al., 1995; Abu-Elheiga et al., 1997; Ha et al., 1996; Thampy et al., 1988; Bianchi et al., 1990) for example, ACC1 is highly expressed in lipogenic tissues such as liver and adipose tissue and that ACC2 is predominantly expressed in heart and skeletal muscle (Thampy et al., 1989; Abu-Elheiga et al., 1995; Bianchi et al., 1990 and Iverson et al., 1990). Both ACC1 and ACC2 produce malonyl-CoA, which is the donor of the “C 2-units” for fatty acid synthesis and the regulator of the carnitine paInitoyl-CoA shuttle system that is involved in the mitochondrial oxidation of long-chain fatty acids (McGarry et al., 1977; McGarry et al., 1997; McGarry et al., 1980). Hence, acetyl-CoA carboxylase links fatty acid synthesis and fatty acid oxidation and relates them with glucose utilization and energy production because acetyl-CoA, the substrate of the carboxylases, is the product of pyruvate dehydrogenase.

Diet, especially a fat-free one, induces the synthesis of ACC's and increases their activities. Starvation or diabetes mellitus represses the expression of the ACC genes and decreases the activities of the enzymes. Earlier studies addressed the overall activities of the carboxylases with specific differentiation between ACC1 and ACC2. Studies on animal carboxylases showed that these enzymes are under long-term control at the transcriptional and translational levels and short-term regulation by phosphorylation/dephosphorylation of targeted serine residues and by allosteric modifications induced by citrate of palmitoyl CoA (Thampy et al., 1988; Kim et al., 1989; Thampy et al., 1988; Mabrouk et al., 1990; Mohamed et al., 1994; Hardie et al., 1989; Hardie et al., 1997). Several kinases have been found to phosphorylate both carboxylases and to reduce their activities. In response to dietary glucose, insulin activates the carboxylases through their phosphorylation. Starvation and/or stress lead to increased glycogen and epinephrin levels that inactivate the carboxylases through phosphorylation (Kim et al., 1989; Thampy et al., 1988; Mabrouk et al., 1990; Mohamed et al., 1994; Hardie et al., 1989; Hardie et al., 1997). Experiments with rats undergoing exercises showed that their malonyl CoA and ACC activities in skeletal muscle decrease as a function of exercise intensity thereby favoring fatty acid oxidation. These changes are associated with an increase in AMP-kinase activity (Hardie et al., 1997; Rasmussen et al., 1997; Winder et al., 1996; and Rasmussen et al., 1999). The AMP-activated protein kinase (AMPK) is activated by a high level of AMP concurrent with a low level of ATP through mechanism involving allosteric regulation and phosphorylation by protein kinase (AMP kinase) in a cascade that is activated by exercise and cellular stressors that deplete ATP (Lopaschuk et al., 1994; Kudo et al., 1995; Dyck et al., 1999; Vavvas et al., 1997). Through these mechanisms, when metabolic fuel is low and ATP is needed, both ACC activities are turned off by phosphorylation, resulting in low malonyl-CoA levels that lead to increase synthesis of ATP through increased fatty acid oxidation and decreased consumption of ATP for fatty acid synthesis.

Obesity is a major health factor that affects the body's susceptibility to a variety of diseases such as heart attack, stroke, and diabetes. Obesity is a measure of the fat deposited in the adipose tissue in response to food intake, fatty acid and triglyceride synthesis, fatty acid oxidation, and energy consumption. Excess food provides not only the timely energy needs of the body, but promotes glycogen synthesis and storage in liver and muscle and fatty acid and triglyceride synthesis and storage in the fat tissues. Calorie restriction or starvation promotes glycogenolysis that supplies glucose where needed and lipolysis that supplies fatty acids for oxidation and energy production. Insulin and glucagon are the hormones that coordinate these processes. Malonyl-CoA is the key intermediate in fatty acid synthesis, and acts as a second messenger that regulates energy levels (ATP) through fatty acid oxidation, which in turn affects fatty acid synthesis and carbohydrate metabolism.

Curcumin and its derivatives are components contained in tropical or subtropical plants, of which a good representative is perennial Curcuma longa, belonging to Zingiberaceae. Curcuma longa is generally known as turmeric, one of spices which are used in curry, and can be used not only for foods, but also as a colorant in food or clothing, or as a herbal medicine in traditional therapies such as Chinese medicine (Kampo), Indian Ayurveda and Indonesian Jamu due to its hemostatic, stomachic, antibacterial and anti-inflammatory actions.

It is known that curcumin has various physiological activities such as anti-oxidative action, cholagogic action, the internal organs (hepatic or pancreatic) function-potentiating action, carcinogenesis-inhibiting action (Ammon et al., 1991; Satoskar et al., 1986; Shankar et al., 1980), lipid metabolism-improving action, and whitening action. In particular, streptozotocin-induced diabetic rats were maintained on diet containing 0.5% curcumin and exhibited reduced cholesterol, triglyceride and phospholipid levels in blood (P. Suresh Babu and K. Srinivasan, 1997) and amelioration of renal lesions associated with diabetes mellitus (P. Suresh Babu and K. Srinivasan, 1998). Japanese Patent Application H11-246399 discloses that enhanced activity of acyl-CoA oxidase (β-oxidation promotive enzyme in the proxisome) and inhibition of triglyceride accumulation in the liver were observed in rats which received curcumm.

Since ACC2 is a key enzyme that modulates the levels of malonyl-CoA, it would be beneficial to develop inhibitors to ACC2 as potential compounds that can be used to promote weight loss and/or treat or prevent obesity. It is known that curcumin has certain physiological actions, however, it is not known that curcumin and/or its analogues can modulate ACC2 activity. Thus, the present invention is the first to describe the use of curcumin and/or its analogues as inhibitors of ACC2 resulting in enhancement of α-oxidation of fatty acids.

### BRIEF SUMMARY OF THE INVENTION

The present invention provides curcumin compositions or compositions of curcumin analogues and methods of using the same, for regulating, modulating or altering lipid metabolism in a manner beneficial to a subject. For example, the curcumin compositions, and methods of using the same, can be used to modulate mitochondrial fatty acid oxidation. In still other embodiments, the present invention provides curcumin compositions, and methods for using the same, to promote weight loss, to treat and/or prevent obesity and obesity-related diseases and/or disorders.

One embodiment of the present invention is a method of increasing mitochondrial fatty acid oxidation comprising contacting a cell with an effective amount of curcumin or analogues thereof. Contacting comprises providing the curcumin or analogues thereof to the cell, in which the effective amount decreases acetyl-CoA-carboxylase 2 (ACC2) activity. A decrease in ACC2 activity promotes fatty acid oxidation in the cell. The effective amount or effective concentration of curcumin or its analogues that is delivered to the cell can be about 1 μM, 5 μM, 10 μM, 15 μM, 20 μM, 25 μM, 30 μM, 35 μM, 40 μM, 45 μM, 50 μM or any range there between. More specifically, the amount can be about 25 μM to about 50 μM.

In certain embodiments of the present invention, the curcumin and/or its analogues are formulated to be administered via an alimentary route. Specifically, the pharmaceutical compositions disclosed herein may be administered orally, buccally, rectally, or sublingually.

In further embodiments, curcumin and/or its analogues may be administered via a parenteral route. Specifically, the pharmaceutical compositions disclosed herein may be administered mucosally, intravenously, intradermally, intramuscularly, transdermally, intraperitoneally, or aerosol particle delivery to the lungs.

Another embodiment of the present invention is a method of promoting weight loss in a subject comprising administering to the subject an amount of curcumin or analogues thereof effective to modulate activity of ACC2. The amount can be administered daily. Modulation of ACC2 activity increases fatty acid oxidation thereby promoting weight loss in the subject and/or modulation of ACC2 activity decreases fatty acid synthesis thereby promoting weight loss in the subject. The amount of curcumin or its analogues that is administered is an amount that results in a blood or plasma concentration of curcumin or its analogues of about 1 μM to about 100 μM, more specifically, 25 μM to about 50 μM.

The subject can be obese or overweight. A subject that is overweight can be one that has an excess of body weight compared to standard height/weight tables, the excess weight can be about 1% to about 20% over the desirable weight for that subject compared to the standard height/weight tables. In certain circumstances, an obese subject can be defined as a subject having at least a 20% or greater increase over desirable relative weight. A more accurate and operational definition of obesity is based on the Body Mass Index (BMI), which is; calculated as body weight per height in meters squared (kg/m 2). Thus, in certain embodiments, an obese subject is one that has a BMI greater than or equal to 27 kg/m², which is considered to be in the 85^{th} percentile for BMI. Thus, an obese subject can be a subject having a BMI greater than or equal to the 85^{th} percentile. An overweight subject can be further defined as subject having a BMI of about 25 kg/m² but lower than 30 kg/m². A “subject at risk of obesity” is an otherwise healthy subject with a BMI of 25 kg/m to less than 30 kg/m² or a subject with at least one obesity-related disease with a BMI of 25 kg/m² to less than 27 kg/m². A subject at risk of obesity may also be considered an overweight subject.

Yet further, another embodiment is a method of modulating mitochondrial fatty acid oxidation in a subject comprising administering to the subject an effective amount of curcumin or analogues thereof. Modulating mitochondrial fatty acid oxidation comprises decreasing ACC2 activity. Still further, modulating is an increase in fatty acid oxidation which results in a decrease in fatty acid synthesis thereby reducing fat accumulation in the subject. An increase in fatty acid oxidation can also promote weight loss in the subject.

A further embodiment of the present invention is a method of treating and/or preventing obesity and/or obesity-related diseases or disorders in a subject comprising administering to the subject an effective amount of curcumin or analogues thereof, wherein said amount modulates mitochondrial fatty acid oxidation. The effective amount of curcumin or analogues thereof is admixed with a pharmaceutical carrier. Modulating mitochondrial fatty acid oxidation comprises decreasing ACC2 activity, which results in an increase in fatty acid oxidation thereby reducing fat accumulation and promoting weight loss.

Obesity-related disease and/or disorders include, but are not limited to hyperinsulinemia, hypertriglyceridemia, hypercholesterolemia, diabetes mellitus (non-insulin dependent or type II), insulin resistance, and hyperlipoproteinemia. Yet further, gross obesity is known to produce mechanical and physical stresses that aggravate and/or cause disorders, including but not limited to osteoarhritis, sciatia, varicose viens, thromboembolism, ventral and hitatal hernias, cholelithiasis, hypertension, hypoventilation syndrome (pickwickian syndrome), and atherosclerosis.

It is envisioned that treatment of obesity and obesity-related disorders using the curcumin compositions of the present invention will reduce or maintain the body weight of an obese subject or a subject at risk of being obese. Treatment may be decreasing the occurrence of and/or the severity of obesity-related diseases, maintaining weight loss, promoting weight loss, an altering metabolic rate, increasing fatty acid oxidation, decreasing fatty acid synthesis, decreasing blood glucose, decreasing insulin, decreasing insulin resistance.

In further embodiments, the curcumin or analogues thereof are administered in combination with another known method for treating and/or preventing obesity, for example, but not limited to a hypocaloric diet or exercise.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the inhibition of ACC2 as a function of curcumin concentration.

### DETAILED DESCRIPTION OF THE INVENTION

It is readily apparent to one skilled in the art that various embodiments and modifications can be made to the invention disclosed in this Application without departing from the scope and spirit of the invention.

I. Definitions

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternative are mutually exclusive, although the disclosure supports a definition that refers to only alternatives and “and/or.”

The term “alimentary route” as used herein is defined as any route that pertains to the digestive tube from the mouth to the anus of the subject. For example, the alimentary route includes, but is not limited to the mouth or buccal cavity, pharynx, esophagus, stomach, small intestine, large intestine or rectum. Exemplary alimentary routes of administration of drugs and/or compositions include, but are not limited to oral, rectal, sublingual or buccal.

The term “analogue” as used herein refers to a natural or synthetic compound that is structurally similar to curcumin.

The term “parenteral” or “parenteral route” as used herein refers to any as route of administration in which the compound is absorbed into the subject without involving absorption via the intestines or the alimentary tract. Exemplary parenteral routes include, but are not limited to intravenous, subcutaneous, intraperitoneal, intramuscular or mucosal. Other parenteral routes include aerosol delivery to the lungs.

The term “overweight” as used herein refers to an excess of body weight compared to standards height/weight tables that are known and used in the art. The excess weight may be from muscle, bone, fat, and/or body weight.

The term “obese” or “obesity” as used herein refers to having an abnormally high proportion of body fat. A body weight 20% over that in standard height-weight tables is arbitrarily considered obesity. Obesity may be classified as mild (20 to 40% overweight), moderate (41 to 100% overweight), or severe (>100% overweight).

The term “subject”, as used herein refers to an animal, preferably a mammal, most preferably a human, who has been the object of treatment, observation or experiment.

The term “subject in need thereof” refers to a subject who is in need of treatment or prophylaxis as determined by one of skill in the art, for example, a researcher, veterinarian, medical doctor or other clinician. In one embodiment, the subject in need of treatment is an obese mammal. In another embodiment, the subject in need of treatment is an obese human with one or more obesity-related diseases and/or disorders. In another embodiment, the subject in need of treatment is an obese human without obesity-related diseases and/or disorders.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated, for example obesity and/or obesity-related diseases.

The term “prophylactically effective amount” as used herein means the amount of the active compounds in the composition that will elicit the biological or medical response in a tissue, system, subject, or human that is being sought by the researcher, veterinarian, medical doctor or other clinician, to prevent the onset of obesity or an obesity-related disorder in subjects as risk for obesity or the obesity-related disorder.

As used herein, “pharmaceutically acceptable carrier” or “pharmaceutical carrier” includes any and all solvents, dispersion media, coatings, surfactants, antioxidants, preservatives (i.e., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

II. Regulation of Fatty Acid Metabolism

Fatty acid metabolism occurs in peroxisomes and mitochondria. In mitochondria, an acyl group from acyl-CoA is transferred across the membrane bilayer by a camitine-dependent transport system. With the exception of the camitine-dependent transport system, mitochondrial and peoxisomal β-oxidation systems carry out basically the same reactions, although with quite different assemblies of enzymes (Lazarow & De Duve 1976). In peroxisomal oxidation, the first reaction is catalyzed by acyl-CoA oxidases and the electrons derived are transferred directly to molecular oxygen (Schulz 1991, Kunau et al. 1995). In contrast, in mitochondria different acyl-CoA dehydrogenases operate jointly with the respiratory chain and channel electrons to ATP generation in oxidative phosphorylation (Williamson & Engel 1984, Ikeda et al. 1985, Izai et al. 1992, Eaton et al. 1996, Eder et al. 1997, Parker & Englel 2000).

Acetyl CoA carboxylase (ACC) is the rate limiting (committed) step in fatty acid synthesis. ACC is activated by citrate and inhibited by palmitoyl-CoA and other long chain fatty acyl-CoAs; and its activity is also affected by phosphorylation. Phosphorylation of ACC occurs through the action of AMP-activated protein kinase, AMPK. Glucagon stimulated increases in PKA activity also results in phosphorylation and inhibition of ACC. Additionally, glucagon activation of PKA leads to phosphorylation and activation of phosphoprotein phosphatase inhibitor-1, PPI-1 which results in a reduced ability to dephosphorylate ACC maintaining the enzyme in a less active state. However, insulin leads to activation of phosphatases, thereby leading to dephosphorylation of ACC, which results in increased ACC activity. Regulation of fat metabolism also occurs through malonyl-CoA induced inhibition of carnitine acyltransferase I. This functions to prevent the newly synthesized fatty acids from entering the mitochondria and being oxidized.

Studies on animal carboxylases, usually a mixture of ACC1 and ACC2, showed that these enzymes are under long-term control at the transcriptional and translational levels and under short-term regulation by phosphorylation/dephosphorylation of targeted Ser residues and by allosteric modifications by citrate or palmitoyl-CoA (McGarry et al., 1977, McGarry et al., 1997, Abu-Elheiga et al., 2000, Alam et al., 1998; Thampy et al., 1988; Bianchi et al., 1990; McGarry et al., 1980; Iverson et al., 1990; Kim et al., 1989; Thampy et al., 1988; Mabrouk et al., 1990; Mohamed et al., 1994; and Hardie et al., 1997; Bressler et al., 1961; Chaudry et al., 1969). Several kinases have been found to phosphorylate both carboxylases and to reduce their activities. Insulin activates the carboxylases through their dephosphorylation, whereas glucagon and epinephrine inactivate them as a result of their phosphorylation (Lopaschuk et al., 1994; Kudo et al., 1995; Dyck et al., 1999; Kim et la., 1989; Mabrouk et al., 1990; Hardie, 1989; Hardie et al., 1997). The AMP-activated protein kinase (AMPK), one of the most notable kinases, is activated by a high level of AMP concurrent to a low level of ATP through mechanisms involving allosteric regulation and phosphorylation by protein kinase (AMPK kinase) in a cascade that is activated by cellular stressors that deplete ATP (Vavvas et al., 1997). Through these mechanisms, when metabolic fuel is low and ATP is needed, both the ACC activities are turned off by phosphorylation, resulting in the low malonyl-CoA levels that lead to increased synthesis of ATP through increased fatty acid oxidation and decreased consumption of ATP for fatty acid synthesis.

The differential expression of ACC1 and ACC2 in various tissues suggest that their functions are different though interrelated. For example, ACC1, which is located in the cytosol) is highly expressed in liver and adipose and ACC2 (which is located on the mitochondrial membrane) is predominant in heart and muscle. The cytosolic ACC1-generated malonyl-CoA is utilized by the fatty acid synthase, which also is a cytosolic enzyme, for the synthesis of fatty acids. The mitochondrial ACC2-generated malonyl-CoA functions as a regulator of CPTI activity—CPTI being the first enzyme that catalyzes the shuttling of long-chain fatty acids into the mitochondria for β-oxidation and energy production. ACC2-generated malonyl-CoA, therefore, is a second messenger that regulates ATP levels through fatty acid oxidation, which, in turn, affects fatty acid synthesis and carbohydrate metabolism.

Thus, it is envisioned in the present invention that modulation of ACC2 can alter fatty acid metabolism to promote weight loss and treat and/or prevent obesity and obesity-related diseases. Such alterations can include decreasing ACC2 activity thereby promoting fatty acid oxidation and limiting fatty acid synthesis. Promotion of fatty acid oxidation can lead to a reduction in fat accumulation resulting in weight loss.

III. Curcumin and Analogues Thereof.

In certain aspects of the present invention curcumin and/or analogues thereof are used as modulators of ACC2 activity. More specifically, the curcumin and/or analogues thereof inhibit or decrease ACC2 activity.

Commercial curcumin includes three major components: curcumin (77%), demethoxycurcumin (17%), and bisdemethoxycurcumin (3%), which are often referred to as “curcuminoids.” As used herein, “curcumin” is defined to include any one or more of these three major components of commercial curcumin, and any active derivative of these agents. This includes natural and synthetic derivatives of curcumin and curcuminoids, and includes any combination of more than one curcumenoid or derivative of curcumin. Analogues of curcumin and curcumenoids include those derivatives or analogues disclosed in U.S. Patent Application Publication 20020019382, Kumar et al., 2000; Mishra et al., 2002; Dinkova-Kostova, 2002; Ohtsu et al., 2002; Ishida et al., 2002; Syu et al., 1998; Sugiyama et al., 1996; Osawa et al., 1995; Naito et al., 2002; Ruby et al., 1995; Rasmussen et al. 2000; Rao et al., 1984; Mukhopadhyay et al., 1982; Rao et al., 1982; Chun et al., 1999; Chun et al., 2002; and Kumar et al., 2003, each of which is herein specifically incorporated by reference.

In certain aspects, 1,7,-bis(4-hydroxy-3-methoxyphenyl)-1,6-heptadiene-3,5-dione is the curcumin that may be used in the present invention. Other curcumin analogues (curcuminoids) that may be used include, for example, demethoxycurcumin, bisdemethoxycurcumin, dihydrocurcumin, tetrahydrocurcumin, hexahydrocurcumin, dihydroxytetrahydrocurcumin, Yakuchinone A and Yakuchinone B, and their salts, oxidants, reductants, glycosides and esters thereof. Such analogues are described in U.S. Patent Application 20030147979; and U.S. Pat. No. 5,891,924 both of which are incorporated in their entirety herein by reference.

Further examples of curcumin analogues include but are not limited to (a) ferulic acid, (i.e., 4-hydroxy-3-methoxycinnamic acid; 3,4-methylenedioxy cinnamic acid; and 3,4-dimethoxycinnamic acid); (b) aromatic ketones (i.e., 4-(4-hydroxy-3-methoxyphenyl)-3-buten-2-one; zingerone; -4-(3,4-methylenedioxyphenyly-2-butanone; 4-(p-hydroxyphenyl)-3-buten-2-one; 4-hydroxyvalerophenone; 4-hydroxybenzylactone; 4-hydroxybenzophenone; 1,5-bis(4-dimethylaminophenyl)-1,4-pentadien-3-one); (c) aromatic diketones (i.e., 6-hydroxydibenzoylmethane) (d) caffeic acid compounds (i.e., 3,4-dihydroxycinnamic acid); (e) cinnamic acid; (f) aromatic carboxylic acids (i.e., 3,4-dihydroxyhydrocinnainic acid; 2-hydroxycinnamic acid; 3-hydroxycinnamic acid and 4-hydroxycinnamic acid); (g) aromatic ketocarboxylic acids (i.e., 4-hydroxyphenylpyruvic acid); and (h) aromatic alcohols (i.e., 4-hydroxyphenethyl alcohol). These analogues and other representative analogues that can be used in the present invention are further described in WO9518606 and WO01040188, which are incorporated herein by reference in there entirety.

Curcumin or analogues thereof may be purified from plants or chemically synthesized using methods well known and used by those of skill in the art. Plant-derived curcumin and/or its analogues can be obtained by extraction from plants including Zingiberaceae Curcuma, such as Curcuma longa (turmeric), Curcuma aromatica (wild turmeric), Curcuma zedoaria (zedoary), Curcuma xanthorrhiza, mango ginger, Indonesian arrowroot, yellow zedoary, black zedoary and galangal. Methods for isolating curcuminoids from turmeric are well known in the art (Janaki and Bose, 1967). Still further, curcumin may be obtained from commercial sources, for example, curcumin can be obtained from Sigma Chemicals Co (St. Louis, Mo.).

Any conventional method can be used to prepare curcumin and its analogues to be used in the present invention. For example, turmericoleoresin, a food additive, which essentially contains curcumin, can be produced by extracting from a dry product of rhizome of turmeric with ethanol at an elevated temperature, with hot oil and fat or propylene glycol, or with hexane or acetone at from room temperature to a high temperature. Alternatively, those can be produced by the methods disclosed in Japanese Patent Applications 2000-236843, H-11-235192 and H-6-9479, and U.S. Patent Application No. 20030147979, which is incorporated by reference herein in its entirety.

In certain embodiments, a purified product of at least one curcumin and/or its analogue may be used. Alternatively, a semi-purified or crude product thereof may be used, provided that it does not contain impurities which may not be acceptable as a pharmaceutical or food product.

IV. Pharmaceutical Formulations

In a preferred embodiment of the present invention, curcumin and analogues thereof are formulated for delivery to a subject and/or cell to modulate or alter ACC2 activity. Thus, curcumin and/or analogues thereof can be dispersed in a pharmaceutically acceptable carrier.

Curcumin is insoluble in water and ether, but is soluble in ethanol, dimethylsulfoxide, and other organic solvents. It has a melting point of 183° C. and a molecular weight of 368.37. A detailed review of the properties and therapeutic potential of curcumin can be found in Aggarwal et al. (2003A), Aggarwal et al. (2003B), and Aggarwal et al. (2003C), each of which is herein specifically incorporated by reference for this section and all other sections of this application.

The preferred dosage of curcumin and/or analogues thereof (also known as the active compound or active component or active composition or active ingredient) may vary depending upon the administration route and the subject's age, weight, medial history, severity of symptoms, etc. Depending upon the dosage and the route of administration, the number of administrations of a preferred dosage or effective amount may also vary according to the response of the subject.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

In further embodiments, the present invention may concern the use of a pharmaceutical lipid vehicle compositions that include curcumin, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a drug in a lipid vehicle. For example, the curcumin may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes. For example, See WO2005020958, which is incorporated herein by reference.

A. Alimentary Compositions and Formulations

In preferred embodiments of the present invention, the curcumin and/or its analogues are formulated to be administered via an alimentary route. Specifically, the pharmaceutical compositions disclosed herein may be administered orally, buccally, rectally, or sublingually.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both. More preferably, gelatin capsules, tablets, or pills are enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells.

Still further, the active compound (curcumin and/or analogues thereof) may be used as a food additive, for example, the composition may be formulated such that it can be sprinkled onto food, admixed in a liquid beverage, etc.

Additional formulations which are suitable for other modes of administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, curcumin and/or its analogues may be administered via a parenteral route. Specifically, the pharmaceutical compositions disclosed herein may be administered mucosally, intravenously, intradermally, intramuscularly, transdermally, even intraperitoneally, or even aerosol particle delivery to the lungs as described in U.S. Provisional App. No. 60/498,135, U.S. Patent Application Publication 20030149113, and U.S. Pat. Nos. 6,613,308; 6,673,843; 6,664,272; 5,401,777; 5,543,158; 5,641,515; and 5,399,363 each specifically incorporated herein by reference in its entirety.

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In other preferred embodiments of the invention, pharmacologically active compositions could be introduced to the subject through transdermal delivery of a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture.

Transdermal administration of the present invention may comprise the use of a “patch”. For example, the patch may supply one or more active substances at a predetermined rate and in a continuous manner over a fixed period of time.

In other embodiments, one may use eye drops, nasal solutions or sprays, aerosols or inhalants in the present invention. The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. (See U.S. Provisional App. No. 60/498,135, U.S. Patent Application Publication 20030149113, and U.S. Pat. Nos. 6,613,308, 6,673,843, 6,664,272, and 5,401,777, each are specifically incorporated herein by reference in its entirety) Suitable propellants include hydrocarbons and hydrocarbon ethers. Suitable containers will vary according to the pressure requirements of the propellant. Administration of the aerosol will vary according to subject's age, weight and the severity and response of the symptoms.

V. Treatment of Obesity and Obesity Related Disorders

In certain embodiments of the preset invention, a composition comprising curcumin and/or curcumin analogues thereof is administered in an effective amount to improve one or more aberrant indices associated with obesity and obesity-related diseases and/or disorders. Obesity-related disease and/or disorders include, but are not limited to hyperinsulinemia, hypertriglyceridemia, hypercholesterolemia, diabetes mellitus (non-insulin dependent or type II), insulin resistance, and hyperlipoproteinemia. Yet further, gross obesity is known to produce mechanical and physical stresses that aggravate and/or cause disorders, including but not limited to osteoarhritis, sciatia, varicose viens, thromboembolism, ventral and hitatal hernias, cholelithiasis, hypertension, hypoventilation syndrome (pickwickian syndrome), and atherosclerosis.

It is envisioned that the subject may be obese. Yet further, the present invention can also be administered to a subject that is at risk of becoming obese, for example, a subject that is overweight, but not considered obese; and/or a subject that has a family history of obesity, but is not yet considered overweight, etc.

Obesity is a condition in which there is an excess of body fat. In certain circumstances, obesity can be defined as a subject having at least a 20 percent or greater increase over desirable relative weight. A more accurate and operational definition of obesity is based on the Body Mass Index (BMI), which is; calculated as body weight per height in meters squared (kg/m²). “Obesity” refers to a condition whereby an otherwise healthy subject has a Body Mass Index (BMI) greater than or equal to 27 kg/m², or a condition whereby a subject with at least one obesity-related disease has a BMI greater than or equal to 27 kg/m². A BMI of about 27 kg/m² is considered to be in the 85^{th} percentile for BMI. Thus, obesity can also be defined as a subject that is greater than or equal to the 85^{th} percentile for BMI. An “obese subject” is an otherwise healthy subject with a Body Mass Index (BMI) greater than or equal to 30 kg/m² or a subject with at least one obesity-related disease with a BMI greater than or equal to 27 kg/m². A “subject at risk of obesity” is an otherwise healthy subject with a BMI of 25 kg/m² to less than 30 kg/m² or a subject with at least one obesity-related disease with a BMI of 25 kg/m² to less than 27 kg/m². An overweight subject can be further defined as subject having a BMI of about 25 kg/m² but lower than 30 kg/m². A “subject at risk of obesity” is an otherwise healthy subject with a BMI of 25 kg/m² to less than 30 kg/m² or a subject with at least one obesity-related disease with a BMI of 25 kg/m² to less than 27 kg/m². A subject at risk of obesity may also be considered an overweight subject.

It is envisioned that treatment of obesity and obesity-related disorders using the curcumin compositions of the present invention will reduce or maintain the body weight of an obese subject or a subject at risk of being obese. Treatment may be decreasing the occurrence of and/or the severity of obesity-related diseases, maintaining weight loss, promoting weight loss, an altering metabolic rate, increasing fatty acid oxidation, decreasing fatty acid synthesis, decreasing blood glucose, decreasing insulin, decreasing insulin resistance.

Another aspect of the present invention comprises using curcumin and/or analogues thereof as a prophylactic treatment or prevention of obesity and obesity-related disorders. Prevention refers to the administration of the compounds or combinations of the present invention to reduce or maintain the body weight of a subject at risk of obesity. Prevention may also include preventing body weight regain of body weight previously lost as a result of diet, exercise, or pharmacotherapy. Another outcome of prevention may be preventing obesity from occurring if the treatment is administered prior to the onset of obesity in a subject at risk of obesity. Yet further, prevention may be decreasing the occurrence and/or severity of obesity-related disorders if the treatment is administered prior to the onset of obesity in a subject at risk of obesity. Another outcome of prevention may be to prolong resistance to weight gain. Another outcome of prevention may be to prevent weight regain. Moreover, if treatment is commenced in already obese subjects, such treatment may prevent the occurrence, progression or severity of obesity-related disorders, such as, but not limited to, arteriosclerosis, Type II diabetes, cardiovascular diseases, osteoarthritis, hypertension, insulin resistance, hypercholesterolemia, hypertriglyceridemia, and cholelithiasis.

In accordance with the present invention, curcumin and/or its analogues is provided in any of the above-described pharmaceutical carriers is administered via an alimentary route and/or parenteral route to a subject suspected of or suffering from obesity and/or obesity-related disease and/or disorders. The precise effective amount of the curcumin composition to be administered is determined by a physician with consideration of individual differences in age, weight, disease severity and response to the therapy. Parenteral routes of administration include, but are not limited to mucosally, intravenously, intramuscularly, or transdermally. Other parenteral routes of administration include, but are not limited to aerosol delivery to the lungs. Alimentary routes of administration include, but are not limited to oral, nasal, buccal, sublingual or rectal. Oral administration of the curcumin composition includes oral, buccal, enteral or intragastric administration. It is also envisioned that the composition is a food additive. For example, the composition is sprinkled on food or added to a liquid prior to ingestion.

An effective amount of the pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease, such as obesity-related diseases. More specifically, the effective amount of the curcumin pharmaceutical composition decreases, reduces, or inhibits ACC2 activity, decreases fatty acid synthesis, increases fatty acid oxidation, decreases fat accumulation, decreases blood glucose, promotes weight loss, etc. Using the methods and compositions of the present invention, one would generally contact a cell with an effective amount of the composition of the present invention. Yet further, to promote weight loss in a subject an effective amount of the curcumin composition of the present invention can be administered to the subject in need of weight loss.

Those of skill in the art realize that depending upon the route of administration, the amount of the composition may vary. For example, the composition may be formulated such that the effective concentration of curcumin or its analogues that is delivered to the cell comprises about 1 μM, 5 μM, 10 μM, 15 μM, 20 μM, 25 μM, 30 μM, 35 μM, 40 μM, 45 μM, 50 μM, 70 μM, 100 μM or any range there between.

A therapeutically effective amount of curcumin or its analogues thereof as a treatment varies depending upon the host treated and the particular mode of administration. In one embodiment of the invention the dose range of the curcumin or its analogues thereof will be an amount that results or achieves a blood or plasma concentration of about 1 μM, 5 μM, 10 μM, 15 μM, 20 μM, 25 μM, 30 μM, 35 μM, 40 μM, 45 μM, 50 μM, 70 μM, 80 μM, 100 μM or any range there between. In specific embodiments, the therapeutically effective amount may be the amount that results in a blood or plasma concentration of curcumin or its analogues thereof in the range about 1 μM to about 100 μM or any range there between, more specifically in a range of about 25 μM to about 50 μM. One of skill in the art is able to determine the blood or plasma levels of curcumin or its analogues by using standard procedures known in the art to measure levels of compounds in the blood or plasma.

Treatment regimens may vary as well, and often depend on the health and age of the patient. The clinician will be best suited to make such decisions based on the known efficacy and toxicity (if any) of the therapeutic formulations such that the administration results in a beneficial pharmaceutical effect.

VI. Combination Treatments

In order to increase the effectiveness of curcumin and/or analogues thereof in the present invention, it may be desirable to combine the curcumin composition of the present invention with other agents/methods effective in weight loss and/or weight management. Therapeutic agents/methods used for treating obesity include hypocaloric diets, exercise, orlistat, amphetamines (methamphetamine, phentermine and phendimetrazine), sibutramine, and topiramate. This process may involve administering the curcumin composition of the present invention and the agent(s) or multiple factor(s) at the same time. This may be achieved by administering a single composition or pharmacological formulation that includes both agents, or by administering two distinct compositions or formulations, at the same time, or at times close enough so as to result in an overlap of this effect, wherein one composition includes curcumin and/or analogues thereof and the other includes the second agent(s).

Alternatively, the composition of the present invention may precede or follow the other treatments, such as exercise, by intervals ranging from minutes to weeks. In embodiments where the other agent and inventive composition are administered or applied separately, one would generally ensure that a significant period of time did not expire between the time of each delivery, such that the agent and the curcumin composition would still be able to exert an advantageously combined effect on weight loss and/or weight management. In such instances, it is contemplated that one may administer both modalities within about 1-14 days of each other and, more preferably, within about 12-24 hours of each other. In some situations, it may be desirable to extend the time period for treatment significantly, however, where several days (2, 3, 4, 5, 6 or 7) to several weeks (2, 3, 4, 5, 6, 7 or 8) lapse between the respective administrations.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Spectrophotometric Assay

The carboxyltransferase activity was measured in a reaction mixture containing 100 mM Tris buffer (pH 8.0), 0.1 mM malonyl-CoA, 10 mM L-malic acid, 0.5 mM NAD+, 0.6 mg/mL BSA, 125 mg malic dehydrogenase, 50 mg citrate synthase, 1-10 mU of ACC enzyme and either 10 mM D-biotin methyl ester or biocytin. The D-biotin methyl ester was not soluble in water and made up as a 50 mM stock solution in 40% (v/v) ethanol, thus making the final ethanol concentration in the assay 8% (v/v). The reaction was initiated by the addition of the biotin carboxyl acceptor. NADH formation was monitored at 340 nm in either 1.0 mL reactions conducted at 30° C. using the Beckman DU640 UV/Vis spectrophotometer (Beckman-Coulter, Fullerton, Calif.) or in 0.2 mL reactions using a UV-transparent microtiter plate with measurements at 30° C. in a SpectraMax 250 microtiter plate reader (Molecular Devices, Sunnyvale, Calif.).

### Example 2

### HPLC Assay

The reaction mixture contained 50 mM Tris buffer (pH 7.5), 6 mM acetyl-CoA, 2 mM ATP, 7 mM KHCO₃, 8 mM MgCl₂, 1 mM DTT, and 1 mg/mL BSA. The reaction was initiated by the addition of citrated-activated ACC (5 mg murine ACC1 or 2.5 mg human ACC2) in a final volume of 0.2 mL and incubated at 30° C. for various times. Reactions were terminated by the addition of 50 mL of 10% perchloric acid, centrifuged for 3 min at 10,000 g and the supernatants analyzed by HPLC for either the production of malonyl-CoA or the consumption of acetyl-CoA over time.

### Example 3

### Radioactive Assay

The reaction mixture contained 50 mM HEPES, pH 7.5, 2.5 mM MnCl₂, 2.0 mM DTT, 0.125 mM acetyl-CoA, 4.0 mM ATP, 12.5 mM [¹⁴C]KHCO₃ (4×10⁶ dpm), 0.75 mg/mL BSA, 10 mM tripotassium citrate and 0.1-0.2 μg ACC enzyme, in a total volume of 150 μL. The reaction was initiated by the addition of ACC2 and the assay was carried out at 37° C. for 2-7 min. The reaction was stopped by the addition of 50 μL of 6 N HCl. Subsequently, 150 μL was transferred into a glass scintillation vial and evaporated to dryness by heating to 85° C. in a heating block for 1 hour. The dried vials were cooled, 0.5 mL of water and 5 mL of ScintiSafe™ 30% were added, and the radioactivity was determined in a Beckman liquid scintillation counter (LS 3801). The tubes, where HCl was added before the addition of acetyl-CoA carboxylase, served as blanks. Avicin G and curcumin inhibitors were made up as stock solutions in water and DMSO, respectively, and added to the assay at the indicated concentrations such that the DMSO concentration was 1% (v/v). All reactions were performed in duplicate. Enzyme activity was based on radioactivity detected in malonyl-CoA and the dpm's set to 100% activity in the absence of any test compound. Inhibition by 1% (v/v) DMSO which was 16% was subtracted from values obtained in the dose response study.

The radioactive assay showed that recombinant human ACC2 activity was detected. Results are shown in Table 1 below, indicating that DMSO did show a measurable inhibition of ACC2 while avicin G alone had no significant effect on ACC2 activity. An equimolar mixture of avicin G and curcumin also exhibited no significant synergistic effect. Curcumin alone exhibited inhibition under these conditions tested.

TABLE 1
Effect of Additives on Human ACC2 Activity

| Additive | ACC Activity (%) ± S.D.^{a} |
|---|---|
| None | 100 |
| 1% (v/v) DMSO | 83 ± 2 |
| 50 μM curcumin^{b} | 57 ± 3 |
| 50 μM avicin G | 106 ± 7 |
| 50 μM curcumin + 50 μM avicin G | 56 ± 6 |

^{a}Activity calculated as described in Material and Methods;
S.D. = standard deviation.
^{b}Curcumin was dissolved in DMSO such that the final DMSO concentration in the assay was 1% (v/v).

### Example 4

### Dose Response Inhibition Patter of Curcumin

A dose-response series was conducted with curcumin (in DMSO) to verify a classical inhibition pattern. In this study, all curcumin concentrations were added in 1% (v/v) DMSO and the enzyme activity measured.

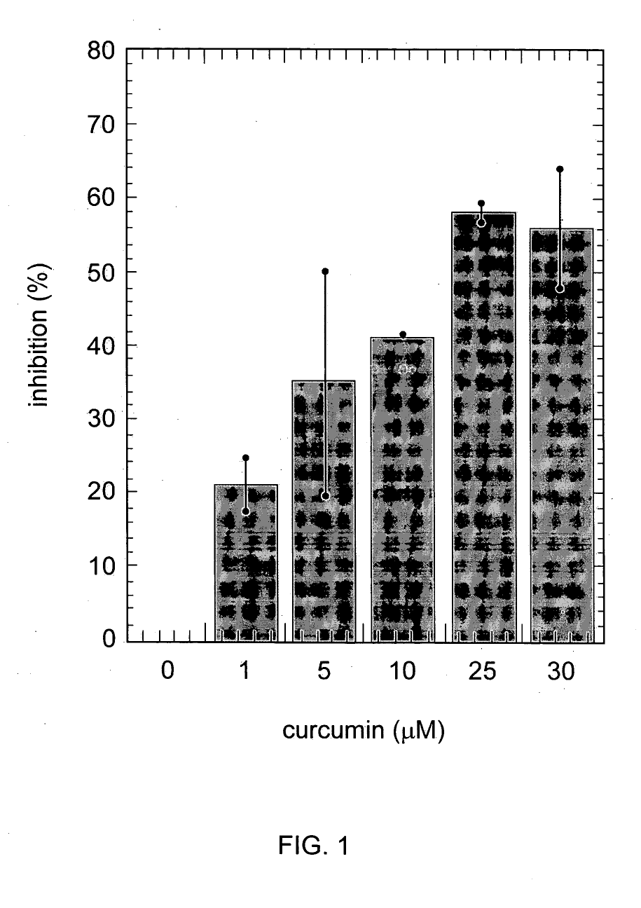

The radioactive assay (as described in Example 3) was used to determine effects of the test compounds, avicin G and curcumin, on human acetyl-CoA carboxylase 2. Curcumin displayed a classical dose-response relationship toward ACC2 inhibition (FIG. 1), indicating that it had some inhibitory effect on ACC2 at the highest physiological concentrations. No significant inhibition was observed for avicin G.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
U.S. Provisional App. No. 60/498,135
U.S. Patent Application Publication 20030149113
U.S. Pat. No. 6,613,308
U.S. Pat. No. 6,673,843
U.S. Pat. No. 6,664,272
U.S. Pat. No. 5,401,777
U.S. Pat. No. 5,543,158
U.S. Pat. No. 5,641,515
U.S. Pat. No. 5,399,363
Japanese Patent Application 2000-236843
Japanese Patent Application H-11-235192
Japanese Patent Application H-6-9479
U.S. Patent Application No. 20030147979
Abu-Elheiga, L., Almarza-Ortega, D. B., Baldini, A., and Wakil, S. J. (1997) J Biol. Chem. 272, 10669-10677.
Abu-Elheiga, L., Jayakumar, A., Baldini, A., Chirala, S. S., and Wakil, S. J. (1995) Proc Natl cad Sci. USA 92, 4011-4015.
Abu-Elheiga, L., W. R. Brinkley, L. Zhong, S. S. Chirala, G. Woldegiorgis, and S. Wakil. (2000)
Aggarwal et al., Anticancer Res., 23(1A):363-398, 2003b.
Aggarwal et al., In: Herbal and Traditional Medicine: Molecular Basis of Health, Packer et al. (Eds.), M. D. Anderson Cancer Center, Texas, 2003a.
Aggarwal et al., In: Phytochemicals in Cancer Chemoprevention, Bagchi et al. (Eds.), M. D. Anderson Cancer Center, Texas, 2003c.
Alam, N., and E. D. Saggerson. (1998) Biochem J. 334:233-41.
Ammon et al., Planta Med., 57:1-7, 1991.
Anderson et al., J. of Immunotherapy, 12:19-31, 1992.
Arbiser et al., Mol. Med., 4:376-383, 1998.
Arun and Nalini, Plant Foods Hum. Nutr. 57:41-52, 2002.
Asai and Miyazawa, J. Nutr., 131:2932-2935, 2001.
Bharti et al., Blood, 101(3):1053-1062, 2003.
Bianchi, A., J. L. Evance, A. J. Iverson, A. C. Nordlund, T. D. Watts, and L. A. Witters. (1990) J Biol. Chem. 265:1502-1508.
Bille et al., Food Chem. Toxicol. 23:967-971, 1985.
Bressler, R. and Wakil, S. J. (1961) J Biol. Chem. 236:1643-1651.
Chaudry, I. H., and M. K. Gould. (1969) Biochem Biophys Acta. 177:527-536.
Chun et al., J. Environ. Pathol. Toxicol. Oncol., 21(2):131-139, 2002.
Chun et al., Mutat. Res., 428(1-2):49-57, 1999.
Conney et al., Adv. Enzyme Regul., 31:385-396, 1991.
Deodhar et al., Indian J. Med. Res., 71:632-634, 1980.
Dikshit et al., Indian J. Med. Res., 101:31-35, 1995.
Dinkova-Kostova, Mini Rev. Med. Chem., 2(6):595-610, 2002.
Dyck, J. R., N. Kudo, A. J. Barr, S. P. Davies, D. G. Hardie, and G. D. Lopaschuk. (1999) Eur J. Biochem. 262:184-190.
Ha, J., J. K. Lee, K.-S. Kim, L. A. Witters, and K.-H. Him. (1996) Proc Natl Acad Sci USA. 93:11466-11470.
Hardie, D. G. 1989. Prog Lipid Res. 28:117-146.
Hardie, D. G., and D. Carling. (1997) Eur J. Biochem. 246:259-273.
Huang et al., Eur. J. Pharmacol., 221:381-384, 1992.
Ibrahimi, A. et al., (1999), J. Biol. Chem., 274:26761-.
Ishida et al., Bioorg. Med. Chem., 10(11):3481-3487, 1998.
Iverson, A. J., A. Bianchi, A. C. Nordlund, and L. A. Witters. (1990) Biochem J. 269:365-371.
Janaki and Bose, An Improved Method for the Isolation of Curcumin From Turmeric, J. Indian Chem. Soc. 44:985 (1967)
Kim, K. H., F. Lopez-Casillas, D. H. Bai, X. Luo, and M. E. Pape. (1989) Faseb J. 3:2250-2256.
Knight et al., Cancer Chemother. Pharmacol., 44(3):177-186, 1999.
Knight, In: Viral and Mycoplasmal Infections of the Respiratory Tract, Lea & Febiger, Philadelphia, 1973.
Korutla and Kumar, Biochim. Biophys. Acta, 1224:597-600, 1994.
Korutla et al., Carcinogenesis, 16:1741-1745, 1995.
Koshkina et al., Cancer Chemother. Pharmacol., 47:451-456, 2001.
Koshkina et al., Cancer Chemotherapy Pharmacol 44: 187-192, 1999.
Koshkina et al., Journal Aerosol Medicine 17:7-14, 2004.
Kudo, N., Bar, A. J., R. L., Desai, S., Lopaschuk, G. D. (1995) J Biol. Chem. 270:17513-17520.
Kumar et al., Biochem. Pharmacol., 55:775-783, 1998.
Kumar et al., Neoplasia, 5(3):255-260, 2003.
Kumar et al., Nucleic Acids Symp. Ser., (44):75-76, 2000.
Kumar et al., Nucleic Acids Symp. Ser., (44):75-76, 2000.
Kuo et al., Biochim. Biphys. Acta, 1317:95-100, 1996.
Kuttan et al., Cancer Lett., 29(2):197-202, 1985.
Li et al., Proc. Natl. Acad. Sci. USA, 90:1839-1842, 1993.
Lim et al., J. Neurosci., 21:8370-8377, 2001.
Limtrakul et al., Cancer Lett., 116:197-203, 1997.
Lopaschuk, G., and Gamble, J. (1994) Can J Physiol Pharmacol. 72:1101-1109.
Lu et al., Carcinogenesis, 15:2363-2370, 1994.
Mabrouk, G. M., Helmy, I. M., Thampy, K. G., and Wakil, S. J. (1990) J. Biol. Chem. 265, 6330-6338.
McGarry et al., (1997) Eur. J. Biochem. 244:114.
McGarry et al., (1977) The Journal of Clinical Investigation.
McGarry et al., (1983) Biochem. J., 214:21-.
McGarry. J. D., and D. W. Foster. (1980) Ann. Rev. Biochem. 49:395-420.
Mehta et al., Anticancer Drugs, 8:470-481, 1997.
Mishra et al., Nucleic Acids Res. Suppl., (2):277-278, 2002.
Mohamed et al., (1994) J Biol. Chem. 269:6859-6865.
Mohan et al., J. Biol. Chem., 275:10405-10412, 2000.
Moss, J. and Lane, M. D., (1971) Adv. Enzymology, 35:321-.
Mukhopadhyay et al., Agents Actions, 12(4):508-515, 1982.
Mukhopadhyay et al., Oncogene, 21(57):8852-8861, 2002.
Naidu and Thippeswamy, Mol. Cell Biochem., 229:19-23, 2002.
Naito et al., J. Atheroscler. Thromb., 9(5):243-250, 2002.
Nandendergche, K., E. A. Rishter, and P. Hespel. (1999) Acta Physiol Scand. 165:307-314.
Natarajan and Bright, J. Immunol., 168:6506-6513, 2002.
Nirmala and Puvanakrishnan, Biochem. Pharmacol., 51:47-51, 1996.
Nirmala and Puvanakrishnan, Mol. Cell Biochem., 159:85-93, 1996.
Nirmala et al., Mol. Cell Biochem., 197:31-37, 1999.
Ohtsu et al., J. Med. Chem., 7:45(23):5037-5042, 2002.
Osawa et al., Biosci. Biotechnol. Biochem., 59(9):1609-1612, 1995.
P. Suresh Babu and K. Srinivasan Molecular and Cellular Biochemistry, 181, 87-96, 1998
P. Suresh Babu and K. Srinivasan, Molecular and Cellular Biochemistry, 166, 169-175, 1997
Pahl, Oncogene, 18:6853-6866, 1999.
Pan et al., Biochem. Pharmacol., 60:1665-1676, 2000.
Piwocka et al., Ann NY Acad. Sci, 973:250-254, 2002.
Piwocka et al., Exp. Cell Res., 249:299-307, 1999.
Plummer et al., Oncogene, 18:6013-6020, 1999.
Punithavathi et al., Br. J. Pharmacol., 131:169-172, 2000.
Quiles et al., Biofactors, 8:51-57, 1998.
Ramachandran and You, Breast Cancer Res. Treat., 54:269-278, 1999.
Ramirez-Tortosa et al., Atherosclerosis, 147:371-378, 1999.
Ranjan et al., J. Surg. Res., 87:1-5, 1999.
Rao et al., Cancer Res., 55:259-266, 1995.
Rao et al., Indian J. Med. Res., 75:574-578, 1982.
Rao et al., Indian J. Physiol. Pharmacol., 28(3):211-215, 1984.
Rasmussen et al., Planta Med., 66(4):396-398, 2000.
Rasmussen, B. B. and Winder, W. W., (1997), J. Appl. Physiol., 83:1104.
Rasmussen, B. B. and Wolfe, R. R., (1999) Am. Rev. Natr. 19:463-.
Ruby et al., Cancer Lett., 94(1):79-83, 1995.
Schwartz, M. W., et al. (1996) Diabetes, 45:531.
Schwartz, M., Erickson, J., Baskin, R., and Palmiter, R. (1998) Endocrinology, 139:2629.
Sidhu et al., Wound Repair Regen., 6:167-177, 1998.
Simon et al., Cancer Lett., 129:111-116, 1998.
Soni and Kuttan, Indian J. Physiol. Pharmacol., 36:273-275, 1992.
Sugiyama et al., Biochem. Pharmacol., 52(4):519-525, 1996.
Syu et al., J. Nat. Prod., 61(12):1531-1534, 1998.
Thaloor et al., Am. J. Physiol., 277:C320-329, 1999.
Thampy, K. G. (1989) J Biol. Chem. 264:17631-17634.
Thampy, K. G., and Wakil, S. J. (1988) J. Biol. Chem. 263, 6454-6458.
Thimmayamma et al., Indian J. Nutr Diet 20:153-162, 1983
Tonnesen and Karlsen, Z Lebensm Unters Forsch., 180(5):402-404, 1985.
Vavvas, D., Apazidis, A., Saha, A. K., Gamble, J., Patel, A., Kemp, B. E., Witters, L. A., and Ruderman, W. B. (1997) J Biol. Chem. 272:13255-13261.
Venkatesan and Chandrakasan, Mol. Cell Biochem., 142:79-87, 1995.
Venkatesan et al., Life Sci., 61:P51-58, 1997.
Venkatesan, Br. J. Pharmacol., 124:425-427, 1998.
Verschraegen, Clin. Cancer Research 10:2319-2326, 2004
Wakil et al., (1983) Ann Rev Biochem. 52:537-579.
Wakil et al., (1958) Biochem. Biopsy. Acta., 29:225.
Waldrep et al., J. Aerosol Med., 7(2):135-145, 1994.
Weibel, In: Morphometry of the Human Lung, Academic Press, NY, 85, 1963.
Winder, W. W. and Hardie, D. G., (1996) Am. J. Physiol., 270:E299.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method of increasing fatty acid oxidation comprising contacting a cell with an amount of curcumin or analogues thereof effective to decrease acetyl-CoA carboxylase activity (ACC2).

2. The method of claim 1, wherein contacting comprises providing said curcumin or analogues thereof to said cell.

3. The method of claim 2, wherein said cell is comprised in a subject.

4. The method of claim 3, wherein the subject is a human.

5. The method of claim 1, wherein said amount that is delivered to the cell is about 25 M to about 50 μM.

6. The method of claim 1, wherein said amount is admixed with a pharmaceutical carrier.

7. The method of claim 1, wherein the amount is administered parenterally.

8. The method of claim 7, wherein parenterally comprises aerosol delivery to the lungs.

9. The method of claim 1, wherein the amount is administered via an alimentary route.

10. A method of promoting weight loss in a subject comprising administering to the subject an amount of curcumin or analogues thereof effective to decrease activity of acetyl-CoA carboxylase activity (ACC2).

11. The method of claim 10, wherein decreasing ACC2 activity increases fatty acid oxidation thereby promoting weight loss in the subject.

12. The method of claim 10, wherein decreasing ACC2 activity decreases fatty acid synthesis thereby promoting weight loss in the subject.

13. The method of claim 10, wherein said subject is obese.

14. The method of claim 10, wherein said subject is overweight.

15. The method of claim 10, wherein said amount is administered daily.

16. A method of modulating fatty acid oxidation in a subject comprising administering to the subject an amount of curcumin or analogues thereof effective to increase fatty acid oxidation.

17. The method of claim 16, wherein an increase in fatty acid oxidation promotes weight loss in the subject.

18. A method of treating and/or preventing obesity in a subject comprising administering to the subject an amount of curcumin or analogues thereof effective to modulate mitochondrial fatty acid oxidation.

19. The method of claim 18, wherein modulating mitochondrial fatty acid oxidation comprises decreasing ACC2 activity.

20. The method of claim 19, wherein a decrease in ACC2 activity results in an increase in fatty acid oxidation.

21. The method of claim 20, wherein said increase in fatty acid oxidation reduces fat accumulation.

22. The method of claim 18, wherein said amount of curcumin or analogues thereof is admixed with a pharmaceutical carrier.

23. The method of claim 18, wherein said curcumin or analogues thereof are administered in combination with another known method for treating and/or preventing obesity.

24. The method of claim 23, wherein said curcumin or analogues thereof administered in combination with a hypocaloric diet or exercise.

25. A method of treating and/or preventing obesity-related disorders in a subject comprising administering to the subject an effective amount of curcumin or analogues thereof, wherein said amount modulates mitochondrial fatty acid oxidation.

26. The method of claim 25, wherein modulating mitochondrial fatty acid oxidation comprising decreasing ACC2 activity.

27. The method of claim 26, wherein a decrease ACC2 activity results in an increase in fatty acid oxidation.

28. The method of claim 27, wherein said increase in fatty acid oxidation reduces fat accumulation.
